(19) United States
(12) Patent Application Publication
(10) Pub. No.: US 2003/0040721 A1
(43) Pub. Date: Feb. 27, 2003

(54) Safety device for a safety syringe

(75) Inventors: Tun-Chi Lee, Hsintien City (TW); Ting Shen, Shanghai (CN)

Correspondence Address: Cooper & Dunham LLP, 1185 Avenue of the Americas, New York, NY, 10036, US

(73) Assignee: Tun-Chi Lee

(21) Appl. No.: 10/223,525

(22) Filed: Aug. 19, 2002

(30) Foreign Application Priority Data
Aug. 27, 2001 (CN) 01253289.4

(51) Int. Cl. A61M 5/00

(52) U.S. Cl. 604/240

(57) ABSTRACT

A safety syringe has a barrel, a needle hub movably received in the barrel and having a hollow cone with a flange formed on an inner periphery of the hollow cone and a plunger with a hook formed to correspond to the flange of the needle hub. The plunger has a spacer formed on the top face of the stop to be adapted to detachably engage with a bottom face of the hollow cone to keep the hook from engagement with the flange. The spacer is made of a material of resilience such that when the spacer is so deformed that the hook engages with the flange so as to pull the needle hub back into the barrel.

### BACKGROUND OF THE INVENTION

1. Field of the Invention

The present invention relates to a safety device for a safety syringe, and more particularly to a safety device which is able to prevent accidental engagement of a hook to a needle hub and to satisfactorily pull the needle hub back into the barrel when required so that the syringe is able to be disposed of safely.

2. Description of Related Art

Numerous patents discussing different structures and means of preventing accidental damage to paramedic personnel by used syringes have already been introduced to the market. Reviewing the patents, most of them concern the means of how the needle is retracted into the barrel. However, in most of the information provided in the available patents, if the user is not careful when trying to receive medicine in the barrel, the plunger prematurely securely engages with the needle hub such that the syringe is no longer useful in injection.

To overcome the shortcomings, the present invention tends to provide an improved safety device for a safety syringe to mitigate and obviate the aforementioned problems.

### SUMMARY OF THE INVENTION

The primary objective of the present invention is to provide an improved safety device for a safety syringe so that prior to giving the injection the user is able to readily pull the needle hub back into the barrel without worrying that the needle hub is accidentally retracted into the barrel.

In order to accomplish the foregoing objective, the syringe has a barrel, a plunger movably received in the barrel and provided with a hook integrally formed with a stop which is formed on a distal end of the plunger and a spacer deformably formed on a top face of the stop, and a needle hub movably received in the barrel and having a flange formed on an inner periphery of the needle hub to correspond to the hook. Engagement between the needle hub and the hook enables the needle together with the needle hub to be readily retracted into the barrel so that accidental damage to the paramedic personnel is prevented. Furthermore, because of the deformability of the spacer, engagement of the spacer with the needle hub will not allow the hook to engage with the flange of the needle hub unless a force is applied to deform the spacer, which triggers engagement between the hook and the flange of the needle hub so that the user is able to use the syringe without accidental engagement of the hook with the flange of the needle hub to disable the syringe.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view showing the parts of the syringe of the present invention;

FIG. 2 is a cross sectional view showing an assembly among the barrel, the needle hub and the plunger;

FIG. 3 is a cross sectional view showing the hook engages with the flange of the needle hub;

FIG. 4 is a cross sectional view showing that the needle hub is retracted back into the barrel by the rearward movement of the plunger; and

FIG. 5 is a schematic view showing that the needle of the needle hub is broken by the forward movement of the plunger.

### DETAILED DESCRIPTION OF THE PREFERRED EMBODIMENT

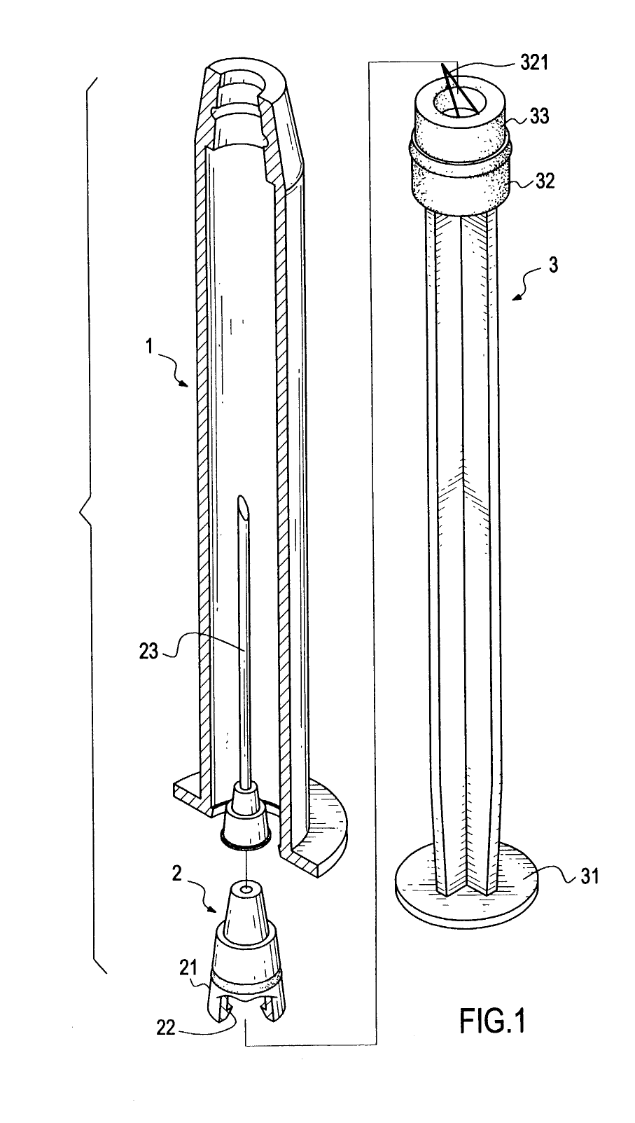

With reference to FIG. 1, a safety syringe has a barrel (1), a needle hub (2) and a plunger (3).

The barrel (1) is hollow inside to receive therein the needle hub (2) and the plunger (3).

The needle hub (2) is detachably received in the barrel (1) and has a hollow cone (21) and a flange (22) formed on an inner periphery of the hollow cone (21) of the needle hub (2). A needle (23) is replaceably mounted on top of the needle hub (2) so that when necessary, the user is able to replace a damaged needle (23) before use.

The plunger (3) has a thumb push (31) formed on a first distal end of the plunger (3) and a stop (32) formed on a second distal end of the plunger (3) and having a hook (321) integrally formed on a top face of the stop (32) to correspond to the flange (22) of the hollow cone (21). The plunger (3) further has a spacer (33) integrally formed on the top face of the stop (32). The spacer (33) is made of a material with resilience such that the spacer (33) is deformable. Furthermore, the hook (321) extends out of the spacer (33) from the stop (32).

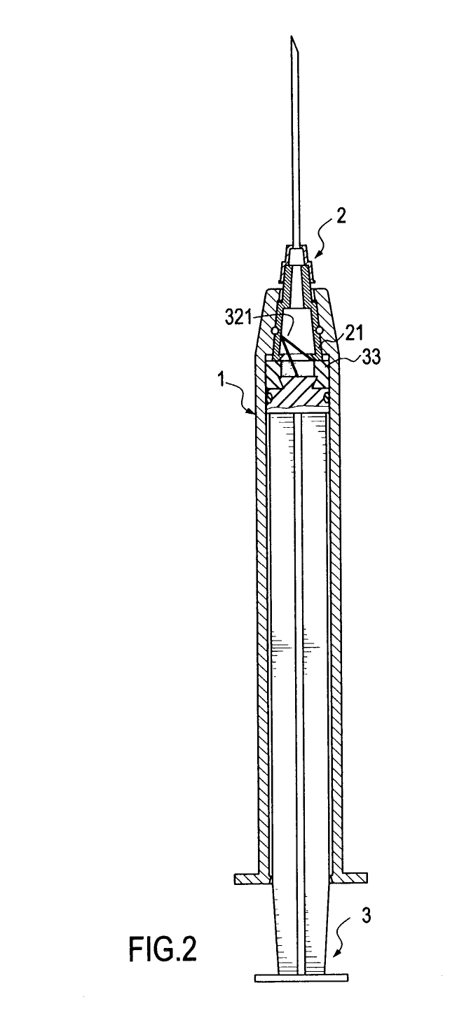

With reference to FIG. 2, when the safety syringe of the present invention is in assembly, the needle hub (2) is first inserted into the barrel (1) so as to secure relative position of the needle hub (2) to the barrel (1). Meantime, the plunger (3) is also received in the barrel (1) to be ready to receive medicine in the barrel (1). From FIG. 2 it is noted that before the syringe of the present invention is in use, the spacer (33) is in engagement with a bottom face of the hollow cone (21) of the needle hub (2) and a free end of the hook (321) is not received in the hollow cone (21), thereby the user can safely use the syringe to receive medicine in the barrel (1) without worrying that the hook (321) might be accidentally in engagement with the needle hub (2) to disable the syringe.

Furthermore, the hook (321) is preferably made of a metal. The hook (321) is bent so that a free end of the hook (321) is resilient relative a distal end securely formed with the stop (32).

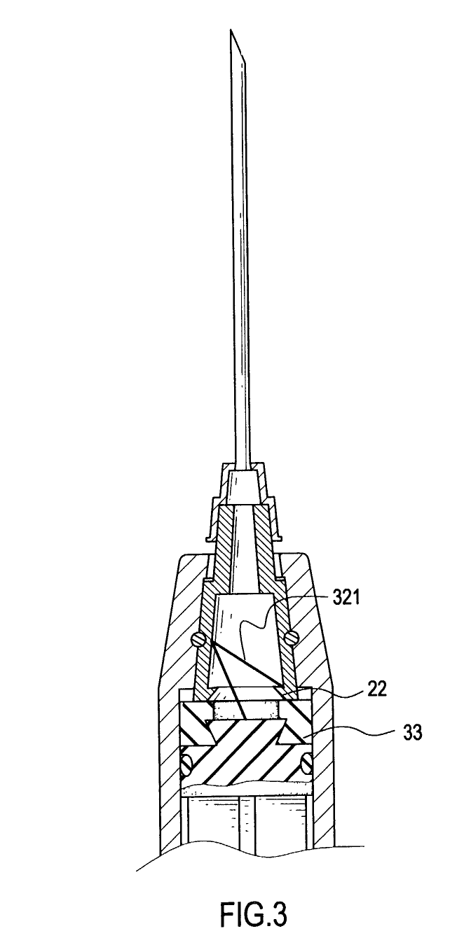

With reference to FIG. 3, when the medicine inside the barrel (1) is ejected out of the barrel (1), the user pushes the plunger (3) all the way into the barrel (1) to force the spacer (33) to deform such that the hook (321) is received in the hollow cone (21). Because of the resilience of the hook (321), the hook (321) is able to engage with the flange (22) of the hollow cone (21). Thereafter the user is ready to pull the needle hub (2) back into the barrel (1).

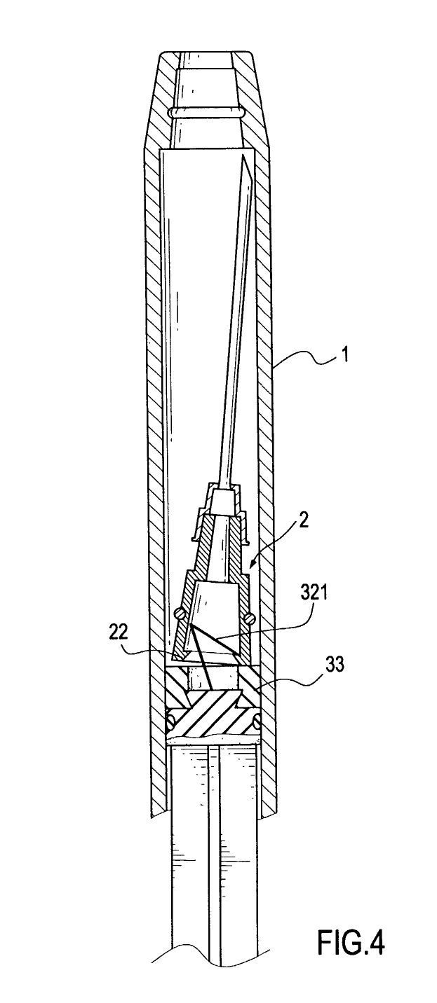

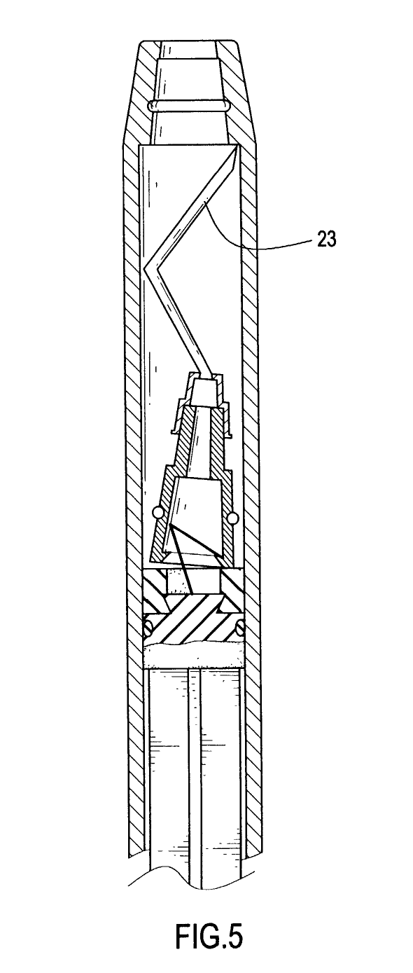

With reference to FIG. 4, after the needle hub (2) is retracted in the barrel (1), because the free end of the hook (321) is in engagement with one point of the flange (22), the needle hub (2) is inclined relative to the barrel (1). When the needle hub (2) is inclined to and fully received in the barrel (1), a forward movement of the plunger (3) together with the needle hub (2), as shown in FIG. 5, the needle (23) is bent inside the barrel (1) such that the safety syringe of the present invention is able to be disposed of safely.

It is thus noted that the safety device used in the safety syringe of the present invention includes the spacer (33) formed on a top face of the stop (32), the hook (321) formed with the stop (32) and extending out from the spacer (33) and a flange (22) formed on the inner periphery of the hollow cone (21) of the needle hub (2).

With such an arrangement of the safety device of the present invention, the user is able to safely use the syringe to perform an injection and readily pull the needle hub (2) together with the needle (23) back into the barrel (1).

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. In a safety syringe having a barrel, a needle hub movably received in the barrel and having a needle securely attached to a distal end of the needle hub and a plunger slidably received in the barrel and having a thumb press formed on a first distal end of the plunger and a stop formed on a second distal end of the plunger, wherein the improvements comprise:
the needle hub has a hollow cone with a flange formed on an inner periphery of the hollow cone;
the plunger has a hook adapted to be integrally formed on a top face of the stop to correspond to the flange of the needle hub so that when the hook engages with the flange of the needle hub, the needle hub is able to be pulled back into the barrel by a rearward movement of the plunger and the needle is damaged by a forward movement of the plunger after the needle hub is entirely received in the barrel and a spacer formed on the top face of the stop to be adapted to detachably engage with a bottom face of the hollow cone to keep the hook from engagement with the flange, the spacer being made of a material of resilience such that when the spacer is so deformed the hook engages with the flange so as to pull the needle hub back into the barrel.
